(19) United States
(12) Patent Application Publication
(10) Pub. No.: US 2025/0314565 A1
(43) Pub. Date: Oct. 9, 2025

(54) TECHNIQUES AND SYSTEMS FOR CREATING SPATIALLY CONTROLLED FLUIDIC FLOWS IN SURFACE FUNCTIONALIZED MICROFLUIDIC DEVICES

(71) Applicant: Xellar Ltd., Grand Cayman (KY)

(72) Inventors: Xin Xie, Lexington, MA (US); Ming Pan, Somerville, MA (US); Xiaohua Qian, Sudbury, MA (US)

(73) Assignee: Xellar Ltd., Grand Cayman (KY)

(21) Appl. No.: 19/092,391

(22) Filed: Mar. 27, 2025

Related U.S. Application Data
(63) Continuation of application No. PCT/US2023/068735, filed on Jun. 20, 2023.
(60) Provisional application No. 63/412,174, filed on Sep. 30, 2022.
(60) Provisional application No. 63/412,273, filed on Sep. 30, 2022.
(60) Provisional application No. 63/412,279, filed on Sep. 30, 2022.
(60) Provisional application No. 63/437,954, filed on Jan. 9, 2023.
(60) Provisional application No. 63/437,955, filed on Jan. 9, 2023.

(51) Int. Cl. G01N 1/36; B01L 3/00; C12M 3/06

(52) U.S. Cl. CPC G01N 1/36; B01L 3/502715; C12M 23/16; B01L 2200/027; G01N 2001/364

(57) ABSTRACT

The present disclosure generally relates to microfluidics, and to spatially controlling fluidic flows. In some embodiments, a fluid in a first microfluidic channel may be prevented from entering a second microfluidic channel due to a trench or other feature separating the channels. Using a trench may avoid the use of pillars, columns, bumps, or other barriers to separate the channels. Thus, for example, a fluid in a first microfluidic channel may be hardened to form a hydrogel, while the second microfluidic channel may remain free of the fluid and the hydrogel. This may allow a barrierless interface between the hydrogel and fluid within the second channel to be formed. Other embodiments are generally directed to devices containing such structures, methods or kits using such structures, or the like.

### RELATED APPLICATIONS

This application claims the benefit of U.S. Provisional Patent Application Ser. No. 63/412,174, filed Sep. 30, 2022, entitled “Microfluidic Devices Containing Hydrogels, and Techniques for Making and Using”; U.S. Provisional Patent Application Ser. No. 63/412,273, filed Sep. 30, 2022, entitled “Methods and Systems for Functionalizing Surfaces for Microfluidic Devices or Other Applications”; U.S. Provisional Patent Application Ser. No. 63/412,279, filed Sep. 30, 2022, entitled “Techniques and Systems for Creating Spatially Controlled Fluidic Flows in Surface Functionalized Microfluidic Devices”; U.S. Provisional Patent Application Ser. No. 63/437,954, filed Jan. 9, 2023, entitled “Edge Effect Systems and Methods for Functionalized Microfluidic Devices”; and U.S. Provisional Patent Application Ser. No. 63/437,955, filed Jan. 9, 2023, entitled “Pipette Interface Systems and Methods for Viscous Fluid Injection.” Each of these is incorporated herein by reference in its entirety.

### FIELD

The present disclosure generally relates to microfluidics, and to spatially controlling fluidic flows.

### BACKGROUND

Microfluidic devices have been used to spatially control fluids in micrometer-sized channels. However, it can be difficult to control the flow of certain kinds of fluids within such microfluidic devices. For example, when a hydrophilic fluid is added to the surface of a hydrophobic thermoplastic material such as polystyrene, the fluid tends to bead up due to surface tension between the two materials. This may create problems, for example, in causing a fluid to flow into desired locations within a microfluidic device. Improvements in systems and methods to control fluid flow of such fluids within microfluidic devices are therefore desirable.

### SUMMARY

The present disclosure generally relates to microfluidics, and to spatially controlling fluidic flows. The subject matter of the present disclosure involves, in some cases, interrelated products, alternative solutions to a particular problem, and/or a plurality of different uses of one or more systems and/or articles.

In one aspect, the present disclosure is generally drawn to an article. In one set of embodiments, the article comprises a substrate defining a first microfluidic channel having a first inlet and a first outlet, and a second microfluidic channel having a second inlet and a second outlet, the first microfluidic channel and the second microfluidic channel positioned parallel and separated by a trench within a common interconnect region positioned between their respective inlets and outlets.

In another set of embodiments, the article comprises a substrate defining a first microfluidic channel and a second microfluidic channel, the first microfluidic channel containing a hydrogel and the second microfluidic channel being free of hydrogel, the first microfluidic channel and the second microfluidic channel positioned parallel within a common interconnect region such that an interface is present within the common interconnect region between the hydrogel in the first microfluidic channel and the second microfluidic channel, the substrate further defining a trench positioned adjacent the interface.

Another aspect is generally drawn to a method. In one set of embodiments, the method comprises providing a substrate defining a first microfluidic channel having a first inlet and a first outlet, and a second microfluidic channel having a second inlet and a second outlet, the first microfluidic channel and the second microfluidic channel positioned parallel within a common interconnect region positioned between their respective inlets and outlets; and passing a fluid through the first microfluidic channel from the inlet towards the outlet, through the common interconnect region, wherein the fluid is prevented from entering the second microfluidic channel via a trench in a wall of the common interconnect region.

In another aspect, the present disclosure encompasses methods of making one or more of the embodiments described herein, for example, microfluidic devices containing trenches or other features for spatially controlling fluidic flows. In still another aspect, the present disclosure encompasses methods of using one or more of the embodiments described herein, for example, microfluidic devices containing trenches or other features for spatially controlling fluidic flows.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIGS. 1A and 1B illustrate microfluidic channels meeting at a common interconnect region, in accordance with certain embodiments;

FIG. 2 illustrates assembly of layers into a substrate, in another embodiment;

FIGS. 3A-3C illustrate microfluidic devices having trenches, in accordance with other embodiments;

FIGS. 4A-4F illustrates a common interconnect region having three microfluidic channels, in yet other embodiments;

FIG. 5 illustrates microfluidic channels meeting at a common interconnect region, in accordance with certain embodiments;

FIGS. 6A-6C illustrate addition configurations of microfluidic devices having trenches, in yet other embodiments; and

FIG. 7 illustrates a microfluidic device having a vent connecting a microfluidic channel to a trench, in still another embodiment.

### DETAILED DESCRIPTION

The present disclosure generally relates to microfluidics, and to spatially controlling fluidic flows. In some embodiments, a fluid in a first microfluidic channel may be prevented from entering a second microfluidic channel due to a trench or other feature separating the channels. Using a trench may avoid the use of pillars, columns, bumps, or other barriers to separate the channels. Thus, for example, a fluid in a first microfluidic channel may be hardened to form a hydrogel, while the second microfluidic channel may remain free of the fluid and the hydrogel. This may allow a barrierless interface between the hydrogel and fluid within the second channel to be formed. Other embodiments are generally directed to devices containing such structures, methods or kits using such structures, or the like.

For example, certain aspects as discussed herein are generally drawn to microfluidic devices that can contain cells, e.g., in contact with a hydrogel or another scaffold medium. For example, cells may be cultured within a microfluidic device, e.g., on or in a hydrogel. The cells may thus be cultured within such a device in an environment that is more similar to their native environment (e.g., where the hydrogel or other scaffold medium may act as an extracellular matrix). In some cases, cells cultured in such conditions may exhibit more physiologically relevant behavior, e.g., due to improved or more biologically relevant cell-to-cell or cell-to-environment interactions. In addition, in certain embodiments, the cells may be cultured in a manner as to emulate various functions of specific organs, e.g., the microfluidic device may be used as an organ-on-a-chip device.

In some embodiments, a hydrogel or another scaffold medium may be contained within a microfluidic device, e.g., within a microfluidic channel defined in a substrate forming the microfluidic device. The hydrogel (or other scaffold medium) may partially or completely fill the microfluidic channel, and cells may be cultured on or in the hydrogel. In addition, in some embodiments, there may be one or more additional microfluidic channels. These may be used for various purposes, e.g., to deliver fluids such as cell media, provide nutrients, remove waste, or the like, to or from the hydrogel. Such channels may be free of hydrogel in certain embodiments. In addition, in some cases such as those discussed below, no physical barrier may be present between the hydrogel and fluid that may be present within the second microfluidic channel.

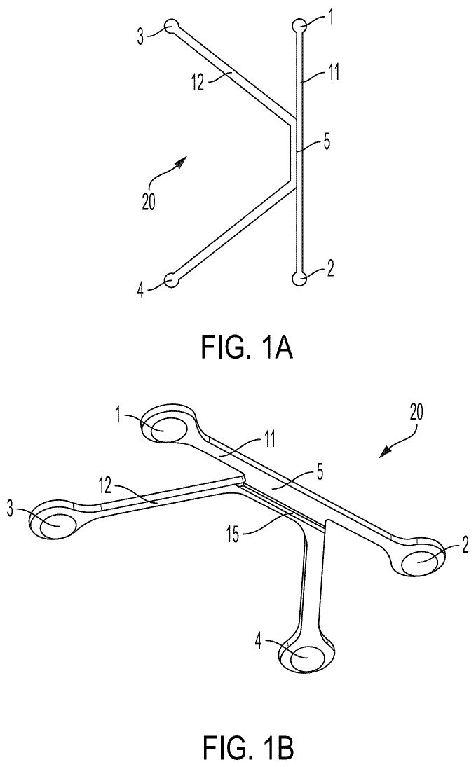

One non-limiting example of such a microfluidic device is shown in FIG. 1A with sample device 20. In this figure, first microfluidic channel 11 connects inlet 1 to outlet 2, while second microfluidic channel 12 connects inlet 3 to outlet 4. First microfluidic channel 11 may be filled with a hydrogel or another scaffold medium, while second microfluidic channel 12 may be empty, e.g., such that during use of the microfluidic device, a fluid (e.g., cell media) can flow from inlet 3 to outlet 4 (or vice versa in some cases). This may be used, for example, to perfuse the cells within the microfluidic device, for example, contained on or within the hydrogel within first microfluidic channel 11.

Also shown in this figure is common interconnect region 5, in which first microfluidic channel 11 and second microfluidic channel 12 come into fluidic contact with each other, e.g., such that a fluid could flow from one channel to the other if both channels were empty. In some cases, both channels may be positioned to be parallel to each other within common interconnect region 5, and in some cases, no physical barrier may be present within common interconnect region 5 that partially or completely separates first microfluidic channel 11 and second microfluidic channel 12 from each other. For example, no pillars, columns, or other barriers may be present that separates first microfluidic channel 11 and second microfluidic channel 12.

In some embodiments, a trench may be positioned between a first microfluidic channel and a second microfluidic channel within a common interconnect region. As discussed in more detail below, the trench may be used to separate or inhibit the flow of fluid from one microfluidic channel to another within the common interconnect region. Such a configuration may allow for separation of fluids to occur within the common interconnect region while avoiding the use of pillars, columns, bumps, phaseguides, ridges, or other barriers that may partially or completely block the common interconnect region. For instance, barriers that at least partially block the first microfluidic channel and the second microfluidic channel may also inhibit the ability of cells to access the cell media (e.g., to access nutrients, remove waste, etc.), and/or make it more difficult to study cells within the microfluidic device, etc., e.g., by making imaging of the cells more difficult. One non-limiting example of such a trench can be seen more easily in FIG. 1B, with trench 15 positioned between first microfluidic channel 11 and second microfluidic channel 12 within common interconnect region 5. However, it should be understood that in other embodiments, a trench may be used in conjunction with ridges, pillars, columns, bumps, phaseguides, or other barriers.

In some embodiments, the trench may be used to separate fluids in one channel (e.g., a first microfluidic channel) from another channel (e.g., a second microfluidic channel). For instance, a fluid flowing through the first microfluidic channel may be inhibited from crossing the trench to reach the second microfluidic channel, e.g., such that the second microfluidic channel remains substantially free of the fluid. In some cases, the volume of fluid flowing through the first microfluidic channel may be controlled, e.g., to help inhibit crossing of the fluid to the second microfluidic channel. Thus, for example, if the first microfluidic channel contains a fluid containing a hydrogel (or another scaffold medium) precursor that is hardened to form a hydrogel, the presence of the trench may prevent the fluid from being able to flow into the second microfluidic channel at the common interconnect region. When the precursor is hardened to form a hydrogel, the hydrogel may be substantively contained within only the first microfluidic channel within the common interconnect region. A fluid flowing in the second microfluidic channel can interact with the hydrogel, without being blocked due to pillars, columns, or other physical barriers. Although other devices have used such physical barriers to separate the fluids in a common interconnect region, such physical barriers often interfere with the ability of fluids in one channel to subsequently interact with another channel within the common interconnect region. In contrast, a trench does not create a physical barrier between the channels.

Accordingly, certain embodiments such as discussed herein are generally directed to microfluidic channels having a polymer or other coating material, and a hydrogel or other scaffold medium, e.g., in contact with the polymer or other coating material. Optionally, cells may be grown on or in the hydrogel, e.g., as discussed herein. Additional non-limiting examples of such devices can be seen in a US provisional patent application, filed on Sep. 30, 2022, entitled “Microfluidic Devices Containing Hydrogels, and Techniques for Making and Using,” U.S. Ser. No. 63/412,174, and techniques for introducing the polymer or other coating material can be seen in a US provisional patent application, filed on Sep. 30, 2022, entitled “Methods and Systems for Functionalizing Surfaces for Microfluidic Devices or Other Applications,” U.S. Ser. No. 63/412,273, each of which is incorporated herein by reference in its entirety. Devices such as these may be used for culturing cells, or other applications such as those discussed herein.

The above discussion is a non-limiting example of certain embodiments that are generally directed to trenches that may be used to separate fluids, e.g., in a common interconnect region. However, other embodiments are also possible. Accordingly, more generally, various aspects of the invention are directed to various systems and methods for spatially controlling fluidic flows, e.g., within microfluidic devices.

One aspect, for example, is generally directed to a microfluidic device, e.g., having one or more microfluidic channels defined in a substrate. The substrate may have any suitable shape or configuration, including square, rectangular, circular, etc. In some cases, the substrate may include one or more layers of material. In certain cases, one or more layers of the substrate may be formed out of materials such as pressure-sensitive adhesives, or other materials, including any of those described herein. For instance, the microfluidic device may include one, two, three, four, or more layers, and one or more of the layers may contain or define one or more microfluidic channels therein. In addition, in some cases, larger channels, tubes, chambers, reservoirs, fluidic pathways, etc. may also be defined within a substrate, e.g., using one or more layers.

The microfluidic channels within the microfluidic device may have any configuration within the device, and there may be one or more than one such channel, which may independently be the same or different. A microfluidic channel may have any cross-sectional shape (circular, oval, triangular, irregular, square or rectangular, or the like) and can be covered or uncovered. The microfluidic channels may be used to move or process fluid within the substrate in any of a number of ways, for example, to allow fluids to flow from one or more inlets, through the microfluidic channel, to one or more outlets.

In some cases, a microfluidic channel may have a maximum cross-sectional dimension of less than 10 mm, less than 8 mm, less than 7 mm, less than 6 mm, less than 5 mm, less than 3 mm, less than 2 mm, and in certain cases, less than 1 mm, less than 500 micrometers, less than 300 micrometers, less than 200 micrometers, less than 100 micrometers, less than 50 micrometers, less than 30 micrometers, less than 20 micrometers, less than 10 micrometers, less than 5 micrometers, etc. In addition, a microfluidic channel may have a maximum cross-sectional dimension of at least 5 micrometers, at least 10 micrometers, at least 20 micrometers, at least 30 micrometers, at least 50 micrometers, at least 100 micrometers, at least 200 micrometers, at least 300 micrometers, at least 500 micrometers, at least 1 mm, at least 2 mm, at least 3 mm, at least 5 mm, at least 6 mm, at least 7 mm, at least 8 mm, at least 10 mm, etc. Any combination of these is also possible. For instance, a microfluidic channel may have a maximum cross-sectional dimension of between 10 micrometers and 30 micrometers, between 100 micrometers and 500 micrometers, between 300 micrometers and 1 mm, or the like.

In some cases, all of the channels within a substrate or a layer may be microfluidic channels. However, in other cases, larger channels, tubes, chambers, reservoirs, fluidic pathways, etc. may also be present. Those of ordinary skill in the art will be familiar with microfluidic channels and systems and methods of making substrates containing microfluidic channels (and/or other channels).

In one set of embodiments, two, three, four, five, or more microfluidic channels may meet at a common interconnect region. In some cases, some or all of the microfluidic channels may be positioned to be parallel to each other within the common interconnect region, and in some cases, no physical barrier (e.g., pillars, columns, bumps, phaseguides, ridges, etc.) may be present within the common interconnect region that partially or completely separates the microfluidic channels from each other. Thus, for example, a fluid could flow from one channel within the common interconnect region to another channel within the common interconnect region if both channels were empty.

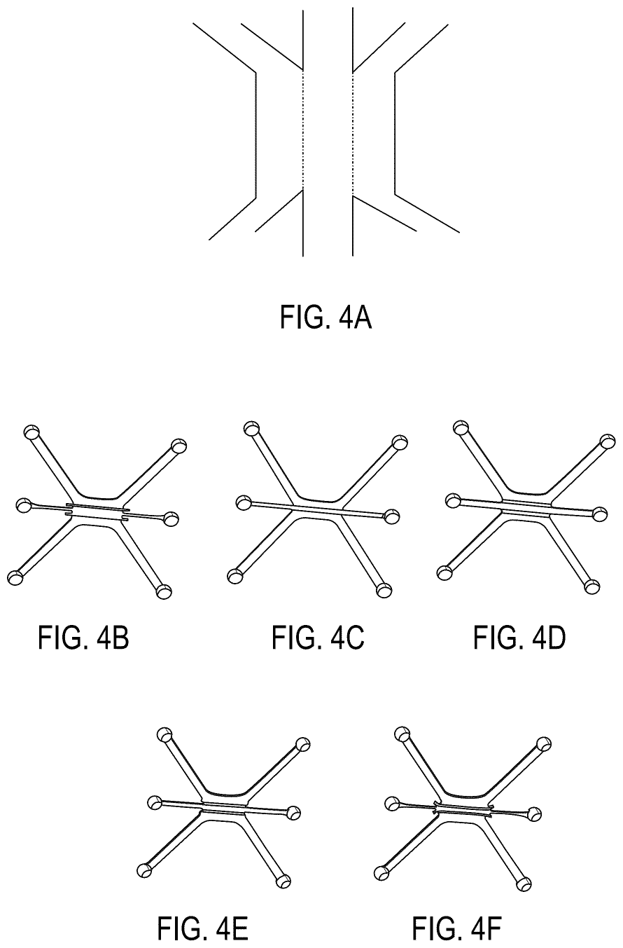

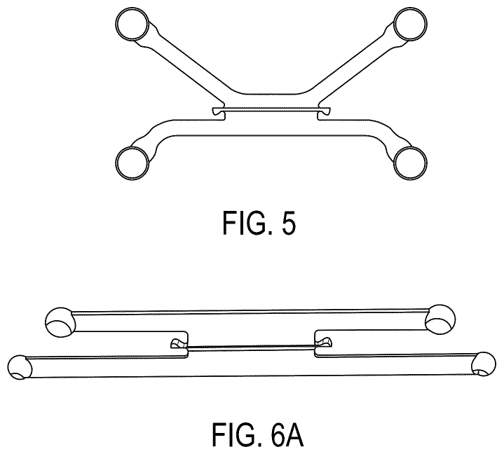

Non-limiting examples of a common interconnect region with two microfluidic channels are shown in FIGS. 1A and 5, while non-limiting examples of common interconnect regions with three microfluidic channels are shown in FIGS. 4A-4F. For example, FIG. 4B shows a common interconnect region with 2 trenches, while FIG. 4C does not have trenches. FIG. 4D illustrates a common interconnect region having ridges present between various microfluidic channels that partially blocks fluidic communication between the microfluidic channels. In addition, combinations of features such as these can be combined in certain embodiments; for example, as is shown in FIGS. 4E and 4F with various embodiments containing both ridges and trenches.

The common interconnect region in some cases, may be treated as a microfluidic channel portion that is composed of two or more microfluidic channels that are in fluidic contact with each other and are generally positioned parallel to each other within the region, although the microfluidic channels may not necessarily be parallel outside of the common interconnect region. In a common interconnect region, the channels are not separated (e.g., by physical barriers such as pillars, columns, bumps, phaseguides, ridges, etc.), and the microfluidic channels can come into contact with each other such that the microfluidic channels in fluidic contact, e.g., to allow fluid flow between channels to occur within the common interconnect region. For example, a first microfluidic channel may have a first inlet and a first outlet, and a second microfluidic channel may have a second inlet and a second outlet, and the first and second microfluidic channels may come into contact and be positioned parallel to each other within the common interconnect region between their respective inlets and outlets (although outside of the common interconnect region, they may or may not also be parallel).

As a non-limiting example, as discussed herein, a first microfluidic channel may contain a hydrogel or other scaffold medium, while a second microfluidic channel may contain a fluid (e.g., cell media), and within the common interconnect region, the fluid is able to come into direct contact with the hydrogel or other scaffold medium, e.g., without having to circumvent a physical barrier, such as a pillar or a column. Accordingly, in certain embodiments, there may be a barrierless interface in a common interconnect region between a first fluid or medium in a first microfluidic channel (for example, a hydrogel or other scaffold medium), and a second fluid or medium in a second microfluidic channel (for example, cell media). For instance, in some embodiments, no interface material or physical barrier separating the first fluid or medium from the second fluid or medium may be present. Thus, for example, a hydrogel or other scaffold medium may partially fill the common interconnect region, for example, such that at least 20%, at least 30%, at least 40%, at least 50%, at least 60%, at least 70%, at least 80%, and/or no more than 80%, no more than 70%, no more than 60%, no more than 50%, no more than 40%, no more than 30%, or no more than 20% of any cross-section of the common interconnect region is not filled with the hydrogel or other scaffold medium. In some embodiments, the hydrogel (or other scaffold medium) partially fills the common interconnect region such that the hydrogel does not prevent bulk fluid flow through at least a portion of the common interconnect region.

In some cases, at least a portion, or all, of the common interconnect region may be substantially straight. In addition, in certain embodiments, the microfluidic channels are positioned within the common interconnect region to be substantially parallel to each other. The parallel microfluidic channels can be used to define an imaginary channel axis that passes through the common interconnect region, e.g., in a direction defined by the direction that the parallel microfluidic channels are oriented. However, in certain cases, one or more of the microfluidic channels may be at an angle relative to other microfluidic channels within the common interconnect region.

In some embodiments, the common interconnect region may have a longest dimension along the channel axis (if present) of at least 1 mm, at least 2 mm, at least 3 mm, at least 4 mm, at least 5 mm, at least 6 mm, at least 7 mm, at least 8 mm, at least 9 mm, at least 10 mm, etc. In addition, the common interconnect region may have a longest dimension along the channel axis of no more than 10 mm, no more than 9 mm, no more than 8 mm, no more than 7 mm, no more than 6 mm, no more than 5 mm, no more than 4 mm, no more than 3 mm, no more than 2 mm, no more than 1 mm, etc. Combinations of these are also possible in other embodiments. For example, the common interconnect region may have a longest dimension of between 5 mm and 7 mm, between 4 mm and 8 mm, between 2 mm and 6 mm, etc.

In certain embodiments, the common interconnect region may have a maximum cross-sectional dimension, or a maximum dimension orthogonal to the channel axis (if present), of at least 100 micrometers, at least 200 micrometers, at least 300 micrometers, at least 500 micrometers, at least 1 mm, at least 2 mm, at least 3 mm, at least 5 mm, at least 10 mm, at least 20 mm, at least 30 mm, at least 50 mm, at least 100 mm, etc. In addition, in certain embodiments, the common interconnect region may have maximum dimensions of no more than 100 mm, no more than 50 mm, no more than 30 mm, no more than 20 mm, no more than 10 mm, no more than 5 mm, no more than 3 mm, no more than 2 mm, no more than 1 mm, no more than 500 micrometers, no more than 300 micrometers, no more than 200 micrometers, no more than 100 micrometers, etc. In addition, combinations of any of these are also possible. For example, a common interconnect region may have maximum dimensions of between 100 micrometers and 300 micrometers, between 5 mm and 10 mm, between 500 micrometers and 2 mm, or the like.

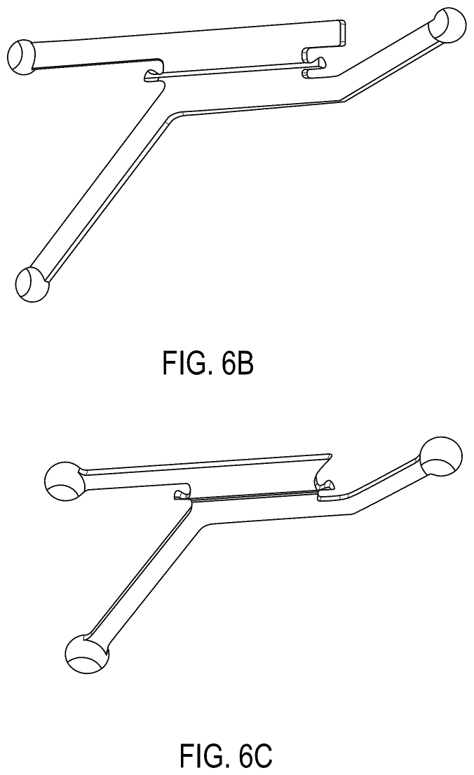

In one set of embodiments, two or more microfluidic channels within a common interconnect region may be separated using a trench, e.g., on or in a wall of the common interconnect region. Additional non-limiting example of trenches are shown in FIGS. 5 and 6.

More than one trench may also be present in some cases, e.g., on opposed surfaces within the common interconnect region. Without wishing to be bound by any theory, it is believed that a fluid flowing in a channel may be attracted to a channel surface, e.g., due to similar hydrophilicities (e.g., if both are relatively hydrophilic or hydrophobic) and/or capillary action, which may facilitate the flow of the fluid within the channel. However, it may be difficult in certain embodiments for such a fluid to be able to cross a trench, e.g., if the volume of fluid is not too great. For example, the trench may exhibit a different hydrophilicity (e.g., one that does not promote attraction with the fluid), and/or the shape of the trench may discourage the fluid from being able to cross, e.g., due to the dimensions of the trench. In some embodiments, the trench may facilitate the flow of fluid through one channel within the common interconnect region, for example, without the fluid flowing into another channel within the common interconnect region. In addition, in certain embodiments, the trench may be treated, e.g., as discussed herein, to render it more hydrophilic or hydrophobic. For example, a coating material, such as a hydrophobic polymer, may be coated on at least a portion of the trench.

Accordingly, in some embodiments, a trench may be positioned within a common interconnect region between a first microfluidic channel and a second microfluidic channel. The trench may run along the length of the common interconnect region in some embodiments, e.g., to separate the two channels. Such a trench may thus provide physical separation of the channels, e.g., without the use of physical barriers (e.g., pillars, columns, bumps, phaseguides, ridges, etc.) to separate the channels. Such trenches are also discussed in more detail in a US provisional patent application, filed on Sep. 30, 2022, entitled “Microfluidic Devices Containing Hydrogels, and Techniques for Making and Using,” U.S. Ser. No. 63/412,174, incorporated herein by reference in its entirety. However, it should be understood that in other embodiments, a trench may be used in conjunction with pillars, columns, bumps, phaseguides, ridges, or other barriers.

The trench may have any suitable dimensions or shape within the common interconnect region. For example, the trench may be substantially straight, or the trench may be bent or curved in certain embodiments. In some cases, the trench may have a length comparable to the length of the common interconnect region. In some embodiments, the trench may have a maximum length of at least 1 mm, at least 2 mm, at least 3 mm, at least 4 mm, at least 5 mm, at least 6 mm, at least 7 mm, at least 8 mm, at least 9 mm, at least 10 mm, etc. In some embodiments, the maximum length may no more than 10 mm, no more than 9 mm, no more than 8 mm, no more than 7 mm, no more than 6 mm, no more than 5 mm, no more than 4 mm, no more than 3 mm, no more than 2 mm, no more than 1 mm, etc. Combinations of these are also possible in other embodiments. For example, the length of the trench may be between 5 mm and 7 mm, between 4 mm and 8 mm, between 2 mm and 6 mm, etc.

In some embodiments, a trench may have a cross-sectional dimension of at least 10 micrometers, at least 20 micrometers, at least 30 micrometers, at least 50 micrometers, at least 100 micrometers, at least 200 micrometers, at least 300 micrometers, at least 500 micrometers, at least 1 mm, at least 2 mm, at least 3 mm, at least 5 mm, at least 10 mm, etc. In addition, in some embodiments, the trench may have a cross-sectional dimension of no more than 10 mm, no more than 5 mm, no more than 3 mm, no more than 2 mm, no more than 1 mm, no more than 500 micrometers, no more than 300 micrometers, no more than 200 micrometers, no more than 100 micrometers, no more than 50 micrometers, no more than 30 micrometers, no more than 20 micrometers, no more than 10 micrometers, etc. In addition, combinations of any of these are also possible, e.g., a trench may have a cross-sectional dimension of between 100 micrometers and 300 micrometers, between 200 micrometers and 1 mm, between 500 micrometers and 3 mm, etc. The trench may have a constant cross-sectional dimension, or a cross-sectional dimension that varies in some embodiments.

In addition, the trench may have any suitable depth. The depth may be independent of the cross-sectional dimension. In some embodiments, the depth may be at least 1 micrometer, at least 2 micrometers, at least 3 micrometers, at least 5 micrometers, at least 10 micrometers, at least 20 micrometers, at least 30 micrometers, at least 50 micrometers, at least 100 micrometers, at least 200 micrometers, at least 300 micrometers, at least 500 micrometers, at least 1 mm, at least 2 mm, at least 3 mm, at least 5 mm, at least 10 mm, at least 20 mm, at least 30 mm, at least 50 mm, etc. In addition, in some cases, the depth may be no more than 50 mm, no more than 30 mm, no more than 20 mm, no more than 10 mm, no more than 5 mm, no more than 3 mm, no more than 2 mm, no more than 1 mm, no more than 500 micrometers, no more than 300 micrometers, no more than 200 micrometers, no more than 100 micrometers, no more than 50 micrometers, no more than 30 micrometers, no more than 20 micrometers, no more than 10 micrometers, no more than 5 micrometers, no more than 3 micrometers, no more than 2 micrometers, no more than 1 micrometer, etc. In addition, combinations of any of these are also possible in certain embodiments. For instance, the trench may have a depth of between 2 mm and 3 mm, between 1 mm and 10 mm, between 100 micrometers and 2 mm, etc. The trench may have a constant depth, or a depth that varies in some cases.

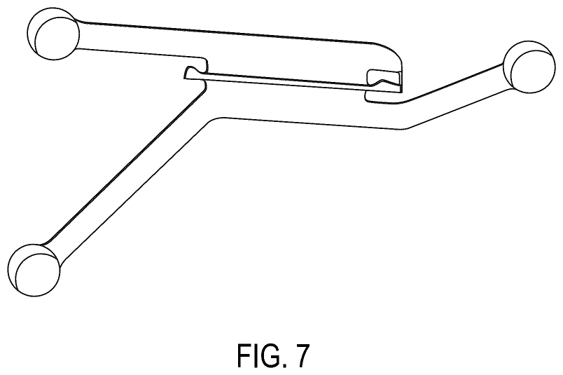

In addition, in one set of embodiments, a microfluidic channel may pass between a single port and a microfluidic interconnect region, e.g., there may not necessarily be both an inlet and an outlet of a microfluidic channel. One example of such a configuration is shown in FIG. 6B. In some cases, a vent may be present at an end of the microfluidic channel, e.g., to allow air or other gases to flow out of the microfluidic channel, for example, when the channel is being filled with a fluid. In some embodiments, the vent may connect an end of the microfluidic channel to a second microfluidic channel, and/or to a trench (if present). One non-limiting examples of such a vent is shown in FIG. 7. However, in other cases, no vent may be present.

In one set of embodiments, the microfluidic channels may have any suitable configuration. If more than one microfluidic channel is present, the channels may independently have the same or different lengths. In some cases, one or more microfluidic channels may intersect, for example, in a T, Y, or a + intersection, or within a common interconnect region such as described herein, etc. Other types of intersections are also possible. A microfluidic channel, in some cases, may be substantially straight between an inlet and an outlet. In addition, in some cases, a microfluidic channel may have one, two, or more bends, curves, or the like between an inlet and an outlet. (As a non-limiting example, as is shown in FIG. 1A, microfluidic channel 12 has two bends between inlet 3 and outlet 4.) If more than one microfluidic channel is present, the microfluidic channels may independently have the same or different configurations. In some cases, there may be 0, 1, 2, or more intersections with other microfluidic channels between an inlet and an outlet of the microfluidic channel.

Non-limiting examples of microfluidic channels with different configurations include those shown in FIGS. 5 and 6A-6C. For instance, in FIG. 6A, two substantially straight microfluidic channels passing between an inlet and an outlet may connect at a common interconnect region, separated by an optional trench in some embodiments.

In addition, it should be understood that a microfluidic channels may not necessarily pass between an inlet and an outlet. For instance, one of the microfluidic channels may have only a single port, which can be used as an inlet and/or an outlet (one non-limiting example is shown in FIG. 6B). In some cases, for instance, fluid may pass through a common interconnect region from an inlet of a first microfluidic channel to an outlet of a second microfluidic channel.

A microfluidic channel may have any suitable pathlength, e.g., length along the channel as a fluid flows between an inlet and an outlet of the channel. If more than one microfluidic channel is present, the microfluidic channels may independently have the same or different pathlengths. For instance, in some embodiments, a microfluidic channel may have a pathlength of at least 1 mm, at least 2 mm, at least 3 mm, at least 4 mm, at least 5 mm, at least 6 mm, at least 7 mm, at least 8 mm, at least 9 mm, at least 10 mm, at least 12 mm, at least 15 mm, at least 20 mm, etc. In some embodiments, the maximum pathlength may no more than 20 mm, no more than 15 mm, no more than 12 mm, no more than 10 mm, no more than 9 mm, no more than 8 mm, no more than 7 mm, no more than 6 mm, no more than 5 mm, no more than 4 mm, no more than 3 mm, no more than 2 mm, no more than 1 mm, etc. Combinations of these are also possible in other embodiments. For example, the length of a microfluidic channel may be between 5 mm and 7 mm, between 4 mm and 8 mm, between 2 mm and 6 mm, etc.

In certain aspects, the microfluidic channels may have any suitable shape, and may connect one or more inlets and one or more outlets. In some cases, such inlets and/or outlets may include ports able to admit a pipette tip. Such ports may be seen, for example, in a U.S. Provisional Patent Application Ser. No. 63/437,955, filed Jan. 9, 2023, entitled “Pipette Interface Systems and Methods for Viscous Fluid Injection,” and a PCT application entitled “Pipette Interface Systems and Methods for Viscous Fluid Injection,” filed on each even date herewith, each incorporated herein by reference in its entirety.

The pipette tip may be, for example, a 1000 microliter pipette tip, a 200 microliter pipette tip, a 10 microliter pipette tip, a 2 microliter pipette tip, or the like. Other sizes are also possible. Many such pipette tips are readily available commercially. In addition, a variety of mechanisms may be used to control fluid in the pipette tip, e.g., to be passed into the microfluidic device. Examples include, but are not limited to, pneumatic pressure or piston-controlled systems, mechanical or manual action, or the like. The pipetting may also be performed manually, or automatically, e.g., using a liquid-handling robot.

The pipette may be inserted into a port of a substrate, such as a microfluidic device. Non-limiting examples of microfluidic devices include any of those described herein, as well as those described in US Pat. Apl. Ser. Nos. 63/412,174, 63/412,273, and 63/412,279, each incorporated herein by reference in its entirety. The port, in one set of embodiments, may be sized so as to admit a pipette tip, e.g., such as any of those described herein. For example, in some embodiments, the port may include an opening having a diameter of less than 10 mm, less than 9 mm, less than 8 mm, less than 7 mm, less than 6 mm, less than 5 mm, less than 4.5 mm, less than 4 mm, less than 3.5 mm, less than 3 mm, less than 2.9 mm, less than 2.8 mm, less than 2.7 mm, less than 2.6 mm, less than 2.5 mm, less than 2.4 mm, less than 2.3 mm, less than 2.2 mm, less than 2.1 mm, less than 2 mm, less than 1.8 mm, less than 1.6 mm, less than 1.5 mm, less than 1.4 mm, less than 1.2 mm, less than 1 mm, less than 0.8 mm, less than 0.7 mm, less than 0.6 mm, less than 0.5 mm, less than 0.4 mm, etc. In addition, in some cases, the opening may have a diameter of at least 0.4 mm, at least 0.5 mm, at least 0.6 mm, at least 0.7 mm, at least 0.8 mm, at least 1 mm, at least 1.2 mm, at least 1.4 mm, at least 1.5 mm, at least 1.6 mm, at least 1.8 mm, at least 2 mm, at least 2.1 mm, at least 2.2 mm, at least 2.3 mm, at least 2.4 mm, at least 2.5 mm, at least 2.6 mm, at least 2.7 mm, at least 2.8 mm, at least 2.9 mm, at least 3 mm, at least 3.5 mm, at least 4 mm, at least 4.5 mm, at least 5 mm, at least 6 mm, at least 7 mm, at least 8 mm, at least 9 mm, etc. Combinations of any of these are also possible in certain embodiments, e.g., the port may have an opening having a diameter of between 2.5 mm and 3 mm, between 2 mm and 2.5 mm, between 4 mm and 4.5 mm, between 2.5 mm and 4 mm, between 2.6 mm and 2.8 mm, between 8 mm and 10 mm, between 0.7 mm and 0.8 mm, between 0.6 mm and 0.7 mm, etc. In addition, in certain embodiments, the port may have an opening that is comparable to the opening of the wells on an ANSI standard microwell-plate, e.g., a 96-well plate, a 384-well plate, or a 1,536-well plate, etc. The opening may be circular, or have other shapes in some cases. If more than one port is present, then the ports may independently be of the same or different sizes.

In some cases, the port may have a diameter or other opening that is larger than that of the cross-sectional dimension of the microfluidic channel, and thus there may be a tapered or funnel region between the microfluidic channel and the port region. The tapering may be linear or non-linear. A non-limiting example of funnel regions are shown in FIG. 5, with funnel regions located between the microfluidic channels and the various ports, which may be used as either inlets or outlets in various embodiments.

However, it should be understood that such funnel regions are not necessarily required, and in some embodiments, there may not be a funnel region between a port and a microfluidic channel in a device. In addition, in some embodiments, some locations in a device may contain such funnel regions, while other locations may not contain such funnel regions.

The opening of the port may allow access to an open portion, which connects to a tapered portion that connects to an end portion in accordance with one set of embodiments. This configuration may be useful to allow a pipette tip entering through the opening to be guided to the end portion, as discussed herein. In one set of embodiments, the open portion is relatively large compared to the size of the pipette tip, and may have a size or dimension that is comparable to the size or dimensions of the opening. The open portion may be substantially cylindrical, or the open portion may be gently tapered in some embodiments.

As mentioned, one set of embodiments, the tapered portion may be sloped so as to guide a pipette tip passing through the opening to be guided into the end portion, and/or so as to allow liquids to flow through the tapered portion into the end portion. Such tapered portions can be fabricated using injection molding techniques, or other techniques such as those described herein. The end portion may have a size or a cross-sectional dimensions that is substantially smaller than the opening of the port, and the tapered portion may connect the two portions. The tapered portion may have a constant slope, or the slope may vary in certain embodiments. In some cases, the tapered portion is circularly symmetric, e.g., about an axis perpendicular to the opening.

As noted, the tapered portion may help to direct the pipette tip into an end portion of the device. The end portion, in one set of embodiments, may be sized so as to allow the pipette tip to fit within, but without too much clearance. For example, the end portion may be sized such that it is difficult for fluid to backflush around the pipette tip, and thus, the fluid is able to flow into an exit to reach microfluidic channels within the device. In addition, in some cases, the clearance between the end portion and the pipette tip may be sufficiently small so as to prevent an excessive amount of fluid remaining within the end portion.

In certain embodiments, the average distance between the pipette tip and the walls of the end portion may be no greater than no greater than no greater than 0.5 mm, no greater than 0.4 mm, no greater than 0.3 mm, no greater than 0.2 mm, no greater than 0.1 mm, no greater than 0.05 mm, etc.

Thus, in some cases, at least 50 vol % of the fluid entering the end portion from the pipette tip may pass through the exit. In some cases, at least 60 vol %, at least 70 vol %, at least 75 vol %, at least 80 vol %, at least 85 vol %, at least 90 vol %, or at least 95 vol % of the fluid entering the end portion from the pipette tip may pass through the exit.

In some embodiments, there may be an exit in contact with the end portion to allow fluid to exit the end portion to reach one or more microfluidic channels. In one set of embodiments, the exit may be in contact with the base of the end portion. In addition, in some embodiments, the exit may be positioned in any suitable location so as to allow fluid from the pipette tip to flow into the microfluidic device, e.g., to reach one or more microfluidic channels such as those disclosed herein.

The exit may be in fluid communication with any of a variety of microfluidic channels in one set of embodiments. The microfluidic channels within the microfluidic device may have any configuration within the device, and there may be one or more than one such channel, which may independently be the same or different. A microfluidic channel may have any cross-sectional shape (circular, oval, triangular, irregular, square or rectangular, or the like) and can be covered or uncovered. The microfluidic channels may be used to move or process fluid within the substrate in any of a number of ways, for example, to allow fluids to flow from one or more inlets, through the microfluidic channel, to one or more outlets.

In addition, in one set of embodiments, a coating material may be present on one or more walls defining a microfluidic channel, for example, to alter the hydrophilicity of the walls. For example, the coating material may increase or decrease the hydrophilicity of at least one of the walls defining a microfluidic channel. Different walls of the microfluidic channel may independently have the same or different hydrophilicities, for example, by coating different walls with different coating materials (or no coating material). Without wishing to be bound by any theory, it should be understood that, due to the small and cramped nature of the microfluidic channels, a fluid within a microfluidic channel may interact with the walls of the microfluidic channels, which can affect the flow properties of the fluid flowing through the channel. Thus, in some embodiments, the hydrophilicities of the walls forming a microfluidic channel may affect the flow of fluid through the channel.

For example, in one set of embodiments, a fluid containing a polymer or other suitable coating material may be flowed through a microfluidic channel, and in some cases, the fluid may be constrained to prevent it from entering other microfluidic channels. For instance, in some cases, a fluid may enter a first microfluidic channel in a common interconnect region, but due to the presence of adhesive or other feature that masks other microfluidic channels within the common interconnect region, the fluid is not able to enter the masked channels. In some cases, the coating material may be deposited onto one or more walls containing the fluid. This may be useful, for example, for altering the hydrophilicity of the walls, for creating a surface for adhering other materials to the walls, for altering the opacity of the walls, or other applications. In addition, other methods of adding a coating material may be used, for example, dip coating or drop casting.

Non-limiting examples of polymers that may be deposited onto one or more walls of a microfluidic channel, e.g., to form a coating thereon, include polyvinylpyrrolidone (PVP), poly(ethylene glycol) (PEG), poly(vinyl alcohol) (PVA), polylysine, or the like. In addition, in some cases, the coating materials may include other materials, in addition to or instead of polymers such as these, for example, ECM attachment factor. In some cases, coating materials, including polymers such as these, may be used to alter or increase the hydrophilicity of the microfluidic channel. In some cases, the increased hydrophilicity may be determined as a change in water contact angle, or by applying 2 microliters of water to a surface of the hydrophilic coating, and measuring a spread of water onto the surface of at least 10 mm².

In one set of embodiments, a hydrogel or other scaffold medium may be positioned on, adjacent to, or attached to the coating, e.g., such that the coating is positioned or located between the hydrogel and a wall of the microfluidic channel. The hydrogel (or other scaffold medium) may be applied, for example, by flowing a fluid containing a hydrogel or other scaffold medium precursor through a microfluidic channel, and treating the precursor to form the hydrogel or other scaffold medium. For example, the hydrogel precursor may be caused to harden to form a hydrogel. In some cases, the fluid containing the precursor may be a hydrophilic fluid, such as water, saline, or buffer, and in certain embodiments, the fluid may be preferentially attracted to a hydrophilic coating material, e.g., that may be present on one or more walls of a microfluidic channel. Examples of hydrophilic coatings include any of those described herein. In some cases, the fluid containing the precursor may preferentially be contained within a first microfluidic channel (e.g., within a common interconnect region as describe herein), without entering other microfluidic channels. Upon treatment (e.g., hardening), the resultant hydrogel (or other scaffold medium) may be positioned on the coating material within the first microfluidic channel, while other microfluidic channels may be substantially free of the hydrogel or other scaffold medium.

Non-limiting examples of hydrogels (e.g., that can be used as an extracellular matrix for cells) include collagen (e.g., Type I collagen, Type II collagen, Type III collagen, etc.), Matrigel®, methacrylated gelatin (Gel-MA), fibrin, alginate, hyaluronic acid, polyacrylamide, poly(ethylene glycol), poly(vinyl alcohol), agarose, agar, chitosan, poly(RADARADARADARADA) (PuraMatrix), poly(AEAEAKAKAEAEAKAK) (EAK16), poly(KLDLKLDLKLDL) (KLD12), or the like. In addition, more than one of these and/or other materials may be present in a hydrogel in certain instances. The collagen may arise from any suitable source, e.g., bovine collagen, rat collagen, fish (marine) collagen, chicken collagen, porcine collagen, sheep collagen, or the like. Other hydrogels will be known by those of ordinary skill in the art. In some embodiments, hydrogels such as these can be formed by flowing a fluid containing a hydrogel precursor, and causing the precursor to form the hydrogel, for example, using a change in temperature (e.g., cooling the device), exposure to ultraviolet radiation, exposure to a chemical, or the like.

In addition, other scaffold media can be used in certain embodiments, e.g., instead of or in addition to a hydrogel as discussed herein. Thus, it should be understood that hydrogels are described herein by way of example only. Non-limiting examples of other scaffold media that may be used in certain embodiments include paraffin, waxes, or the like. These may be added, for example, by flowing a fluid containing an scaffold medium precursor into a microfluidic channel within the device, and treating the precursor to form the scaffold medium within the device. For example, a paraffin or a wax may be introduced into a device at a temperature where the material is liquid, and treated (e.g., cooled) to solidify the medium within the microfluidic device.

In addition, in one set of embodiments, the scaffold medium may be substantially transparent, e.g., to allow for imaging of cells, such as is described herein. As a non-limiting example, in one embodiment, a hydrogel comprising collagen may be used.

According to one set of embodiments, the hydrogel or other scaffold medium may be exposed to cells, which may be grown or cultured on or in the hydrogel or other scaffold medium in some embodiments. Any suitable technique may be used to apply the cells. In some cases, for instance, the cells may be suspended in solution, which is flowed past the hydrogel or other scaffold medium, e.g., within the common interconnect region, and allowed to incubate there to promote attachment of the cells. In some cases, this process may occur over a period of at least 24 hours, or other suitable times. In addition, in some cases, the cells may be mixed with a fluid containing a hydrogel precursor or other scaffold medium precursor, e.g., prior to introduction to the microfluidic device. The cells may then be incubated and allowed to become embedded within the hydrogel or other scaffold medium. Those of ordinary skill in the art will be familiar with techniques for attaching cells to a suitable scaffold medium. Without wishing to be bound by any theory, it is believed that culturing cells on or in such an scaffold medium, e.g., a hydrogel, may more closely approximate the conditions that the cells naturally grow in, e.g., as opposed to a 2-dimensional surface. Accordingly, such cells may respond more similarly and appropriately when cultured in a 3-dimensional environment, such as a hydrogel.

Examples of cells that may be cultured on or in a hydrogel or other scaffold medium include, but are not limited to, mammalian cells such as human cells. Specific non-limiting examples include fibroblasts, lung cells, liver cells, fat cells, kidney cells, intestinal cells, brain cells, epithelial cells, endothelial cells, stromal cells, immune cells, or the like. In some cases, the cells may be stem cells, such as pluripotent stem cells, totipotent stem cells, multipotent stem cells, etc. Other cell types are also possible. In some cases, more than one type of cell may be present, e.g., liver cells and fibroblasts. In addition, in certain embodiments, the cells may produce organoids, tubes, or other 3-dimensional structures, e.g., depending on the cells being cultured.

In some cases, the cells may be cultured within the microfluidic device, for example, within a common interconnect region. In some cases, for instance, in a common interconnect region, a first microfluidic channel may contain a hydrogel or other scaffold medium, and cells that are in contact with the hydrogel or other scaffold medium. The common interconnect region may also comprise a second microfluidic channel that can contain a fluid (for example, cell media) that is able to maintain the cells within the hydrogel. Non-limiting examples of cell media include MEM, DMEM, RPMI, IMDM, F-10, or the like. Those of ordinary skill in the art will be able to select appropriate cell media, e.g., based on the type of cells that are present within the common interconnect region. In some cases, fluid is able to flow in and out of the common interconnect region, e.g., as the hydrogel (or other scaffold medium) may only partially fill the common interconnect region, thereby allowing fluid flow to occur through the common interconnect region. In addition, in some cases, the fluid may be in direct contact with the hydrogel or other scaffold medium, e.g., without having to circumvent a pillar, column, or other physical barrier. Thus, in some embodiments, there may be a barrierless interface between the hydrogel or other scaffold medium and a fluid (e.g., cell media) within the common interconnect region. This may allow the cells to be perfused by the cell media, e.g., to provide nutrients or dissolved gases, remove waste, or the like.

In addition, according to one set of embodiments, a first microfluidic channel and a second microfluidic channel may meet at a common interconnect region where the channels are positioned parallel within the common interconnect region. As previously discussed, there may optionally be a trench positioned between the first microfluidic channel and the second microfluidic channel at the common interconnect region. In some cases, the first microfluidic channel may be a straight channel between a first inlet and an outlet, while the second microfluidic channel may include bends on either side of the common interconnect region between a second inlet and a second outlet, thereby forming a K-shaped structure. A non-limiting example of such a structure can be seen in FIG. 1A. In some cases, as discussed herein, one or more of the channels may contain a hydrogel or other scaffold medium, e.g., such that the hydrogel or other scaffold medium does not completely fill the common interconnect region and a fluid can pass between an inlet and an outlet through a microfluidic channel within the common interconnect region, e.g., in a microfluidic channel that is free of the hydrogel or other scaffold medium.

In one set of embodiments, there may be a plurality of repeat units on a substrate, e.g., repeat units including one or more microfluidic channels or common interconnect regions, such as those described herein. For instance, there may be at least 3, at least 5, at least 10, at least 20,at least 30, at least 40, at least 50, at least 60, at least 70, at least 80, at least 90, at least 100, etc. repeat units on a substrate. The repeat units may be all identically oriented, or they may be differently oriented (e.g., rotated, flipped, etc.) in certain embodiments. In addition, in some cases, two, three, or more types of repeat units may be present on a substrate, e.g., having dissimilar configurations.

In some embodiments, the repeat units may be regularly arranged on a substrate. For instance, the repeat units may be arranged as a square, a rectangle, a circle, a hexagonal configuration, or the like. In addition, the repeat units may be irregularly arranged in certain cases. As an example, the repeat units may be arranged in a 2×n configuration, where n is 1, 2, 3, 4, 5, 6, 7, 8, 9, 10, or the like. As another non-limiting example, the repeat units may be arranged in a 3×n configuration, a 4×n configuration, a 6×n configuration, an 8×n configuration, a 12×n configuration, a 16×n configuration, or the like. For example, the repeat units may be arranged in a 6×6 configuration, an 8×8 configuration, or the like, a 16×16 configuration, or the like.

The microfluidic channels, according to one set of embodiments, may be contained with a substrate having dimensions comparable to a microscope slide, e.g., arranged into a plurality of repeat units on the substrate. For example, the substrate may have dimensions of 75 mm×25 mm, 75 mm×26 mm, 46 mm×28 mm, 46 mm×27 mm, 75 mm×38 mm, 76 mm×51 mm, 76 mm×52 mm, etc. In some cases, such dimensions may vary somewhat (for example, by +/−1 mm, +/−2 mm, or +/−5 mm, etc.), e.g., to allow for manufacturing tolerances or the like. Such dimensions may be useful in some embodiments, e.g., to interface with laboratory equipment able to handle microscope slides.

In another set of embodiments, the microfluidic channels may be contained with a substrate having dimensions comparable to a microwell plate, e.g., one having ANSI dimensions of 128 mm×85 mm, e.g., arranged into a plurality of repeat units on the substrate. In some cases, the dimensions may vary somewhat (for example, by +/−1 mm, +/−2 mm, or +/−5 mm, etc.), e.g., to allow for manufacturing tolerances or the like. Such dimensions may be useful in some embodiments, e.g., to interface with laboratory equipment, such as plate readers or liquid handling robots that are able to handle microwell plates. In addition, in some embodiments, one or more inlets and/or outlets may be positioned within the substrate to match the locations of wells on a microwell plate, e.g., the center locations of the wells on a 24-well standard microplate, a 48-well standard microplate, a 96-well standard microplate, a 384-well standard microplate, or a 1536-well standard microplate, etc.

The substrate may be formed from any suitable materials. In some cases, the substrate may be formed from one, two, three, four, five, or more layers of materials, which may independently be the same or different. For instance, a layer within the substrate may comprise glass or a polymer. Non-limiting examples of polymers include polystyrene, polycarbonate, polymethylmethacrylate (PMMA), polycarbonate, polypropylene, cyclic olefin polymers (COP), cyclic olefin copolymers (COC), polyethylene terephthalate (PET), or the like. For example, an outer or end layer of the substrate may comprise glass or polymer, which may be useful for protecting internal components of the microfluidic device. In addition, as discussed herein, one or more of the layers of the microfluidic channel may be chosen to be substantially transparent.

In addition, in one set of embodiments, the substrate may include a layer comprising a pressure-sensitive adhesive (PSA). In some cases, a layer may be formed from a PSA. Non-limiting examples of pressure-sensitive adhesives include acrylic-based adhesives, silicone-based adhesives (e.g., polydimethylsiloxane), polyurethane-based adhesives, or the like. Certain PSAs may be readily obtainable commercially. In some embodiments, pressure-sensitive adhesives may be particularly useful for defining one or more features, such as microfluidic channels or other channels, tubes, chambers, reservoirs, fluidic pathways, trenches, or the like, e.g., as discussed herein. In some embodiments, for example, one or more features may be defined within a pressure-sensitive adhesive layer, e.g., using cutting techniques such as laser cutting, die cutting, or the like. In some cases, such features may be removed from the pressure- sensitive adhesive, thereby defining the feature within the pressure-sensitive adhesive. In addition, in some cases, such pressure-sensitive adhesives may be pressed or adhered onto another layer, e.g., to form a microfluidic device. For example, in one set of embodiments, a pressure-sensitive adhesive layer may be sandwiched between two other layers (which may be compositionally the same, or different). In addition, in some embodiments, more than one pressure-sensitive adhesive may be present within a microfluidic device.

In some cases, the substrate, or one or more layers, may be chosen to be substantially transparent, for example, to allow for imaging of the common interconnect region (for example, cells within the common interconnect region), or other locations within the substrate. In some embodiments, the entire substrate may be substantially transparent. A variety of techniques may be used for imaging, including light or optical microscopy, confocal microscopy, fluorescence microscopy, microwell plate readers, or the like. Those of ordinary skill in the art will be aware of other suitable imaging techniques. In some cases, multiple locations within a microfluidic device may be studied, e.g., sequentially and/or simultaneously. For example, in some embodiments, the microfluidic device may contain a plurality of repeat units that can be independently determined. In certain embodiments, fluid (e.g., cell media) may be flowed through a common interconnect region (e.g., to perfuse cells, etc., as discussed herein) during imaging (for example, uni-or bidirectionally), although in other cases no such flow may occur during imaging.

In one set of embodiments, microfluidic devices such as those described herein may be used for the study of cells or other constructs, such as organoids, tubes, or other 3-dimensional structures. These may be present, for example, in a common interconnect region, such as is described herein. In some cases, for example, the cells may act as an organ, e.g., the cells may be able to emulate one or more functions of a specific organ. In some embodiments, microfluidic devices having such cells or other constructs may be used to study their function, for example, microscopically (e.g., using imaging such as discussed herein), and/or by analyzing media exiting the microfluidic device (e.g., after being exposed to the cells or other constructs), etc. For example, fluid (e.g., cell media) exiting the microfluidic device may be studied to determine proteins, enzymes, nucleic acids, nutrients, waste gases, or the like, e.g., after exposure to the cells or other constructs.

In addition, in some cases, microfluidic devices having such cells or other constructs may be used to determine the effects of agents thereon. For example, cells or other constructs contained within a microfluidic device (e.g., in a common interconnect region) may be exposed to one or more agents that are suspected of being able to interact, and in some cases alter, such cells or other constructs. The agent may be, for example, a pharmaceutical, a drug, a toxin, a biomolecule, or the like. The agent may be supplied to the cells or other constructs, e.g., separately, or along with cell media that is introduced to the microfluidic device. One or more agents may be used.

In addition, in some cases, as discussed, a microfluidic device may contain more than one such system, e.g., as in a plurality of repeat units on a substrate. In some cases, multiple experiments may be performed simultaneously, e.g., exposure to different agents, and/or the same agents at different concentrations, control experiments, etc., may be performed using different repeat units within the microfluidic device. These experiments may be arranged, e.g., systematically or randomly within the microfluidic device.

In addition, certain aspects are generally directed to methods of making microfluidic devices such as those described herein. Additional techniques for making microfluidic devices include those described in a US provisional patent application, filed on Sep. 30, 2022, entitled “Methods and Systems for Functionalizing Surfaces for Microfluidic Devices or Other Applications,” U.S. Ser. No. 63/412,273, incorporated herein by reference.

For example, in one set of embodiments, a microfluidic device may be formed from a first layer (e.g., a relatively hydrophobic polymer such as polystyrene) and a second layer (e.g., a pressure-sensitive adhesive). In some cases, the second layer may be pressed onto the first layer to form a substrate. In some cases, the second layer may be pre-cut (e.g., laser-cut) with one or more microfluidic channels, or other suitable channels, chambers or fluidic pathways, etc. After adhesion, at least a portion of the second layer may be removed, e.g., to define a suitable channel or other fluidic pathways.

In some cases, the exposed portions of the first layer and/or the second layer may be treated with a polymer or other coating material, e.g., to render them more hydrophilic. Thus, for example one or more walls defining a microfluidic channel may be partially or fully coated with a polymer or other coating material. In addition, in some cases, before a coating material is added, one or more of the surfaces (e.g., of the microfluidic channel) may be treated to facilitate the addition of the coating material. Non-limiting examples of suitable surface treatments include oxygen plasma treatment, corona plasma treatment, or the like.

The polymer or other coating material may be added to the exposed portions using any suitable technique. Examples of suitable polymers include PVP, PEG, PVA, or other polymers such as those described herein. For example, a fluid containing the polymer (or other coating material) may be added to the exposed portions, e.g., by flowing from an inlet to an outlet of a microfluidic channel, and the polymer may be able to coat the exposed surfaces (for example, portions of the surface that had been surface treated as discussed above). In some cases, after waiting for a suitable period of time, the fluid containing the polymer may also be removed, thereby resulting in coated portions within the microfluidic channels.

In some cases, after treatment, other portions (e.g., portions that may have been pre-cut with one or more microfluidic channels, or other suitable channels, chambers or fluidic pathways, etc.) may be removed from the second layer, thereby resulting in a microfluidic device having channels with different hydrophilicities.

In one set of embodiments, optionally, an additional, third layer may be added on top to close the microfluidic channels, e.g., to produce the final microfluidic device. The third layer may, for example, be a polymer layer, and it may be the same or different from the first layer of the device. In some cases, the third layer may include one or more ports or holes to define inlets and/or outlets, for example, to allow fluids to flow into and/or out of the device, e.g., through one or more microfluidic channels.

In some embodiments, a fluid may be passed through microfluidic channels within the device. In some cases, such a fluid may contain a precursor of a hydrogel or other scaffold medium, which may be treated (e.g., hardened) to form a hydrogel or other scaffold medium. In some cases, the hydrogel or other scaffold medium may be formed on the polymer or other coating material within a microfluidic channel, which may be more hydrophilic and allow the fluid to contact and readily flow through the microfluidic channel. Thus, certain embodiments such as discussed herein are generally directed to microfluidic channels having a polymer or other coating material, and a hydrogel that is in contact with it, e.g., such that the polymer is positioned between the hydrogel (or other scaffold medium) and one or more walls of the microfluidic channel.

In addition, in certain embodiments, the hydrogel (or other coating material) may be substantively contained within a microfluidic channel, e.g., within a common interconnect region having other microfluidic channels, for example, without the hydrogel being blocked due to pillars, columns, bumps, phaseguides, ridges, or other physical barriers. However, it should be understood that in other embodiments, a hydrogel (or other coating material) may be used in conjunction with pillars, columns, bumps, phaseguides, ridges, or other barriers.

The following are each incorporated herein by reference in their entireties: U.S. Provisional Patent Application Ser. No. 63/412,174, filed Sep. 30, 2022, entitled “Microfluidic Devices Containing Hydrogels, and Techniques for Making and Using”; U.S. Provisional Patent Application Ser. No. 63/412,273, filed Sep. 30, 2022, entitled “Methods and Systems for Functionalizing Surfaces for Microfluidic Devices or Other Applications”; U.S. Provisional Patent Application Ser. No. 63/412,279, filed Sep. 30, 2022, entitled “Techniques and Systems for Creating Spatially Controlled Fluidic Flows in Surface Functionalized Microfluidic Devices”; U.S. Provisional Patent Application Ser. No. 63/437,954, filed Jan. 9, 2023, entitled “Edge Effect Systems and Methods for Functionalized Microfluidic Devices”; and U.S. Provisional Patent Application Ser. No. 63/437,955, filed Jan. 9, 2023, entitled “Pipette Interface Systems and Methods for Viscous Fluid Injection.”

In addition, the following patent applications, filed on even date herewith, are incorporated herein by reference in their entireties: a PCT application entitled “Microfluidic Devices Containing Hydrogels, and Techniques for Making and Using”; a PCT application entitled “Methods and Systems for Functionalizing Surfaces for Microfluidic Devices or Other Applications”; a PCT application entitled “Edge Effect Systems and Methods for Functionalized Microfluidic Devices”; and a PCT application entitled “Pipette Interface Systems and Methods for Viscous Fluid Injection.”

Furthermore, the following patent applications, filed on even date herewith, are incorporated herein by reference in their entireties: a US design application entitled “Fluid Channel”; a US design application entitled “Fluid Channel Trench”; a US design application entitled “Well Plate”; a US design application entitled “Fluid Channel”; a US design application entitled “Sample Plate”; and a US design application entitled “Sample Plate Carrier.”

The following examples are intended to illustrate certain embodiments of the present disclosure, but do not exemplify the full scope of the disclosure.

### Example 1

This example describes a method to functionalize and partition microfluidic chip surfaces in order to form an ECM channel where an extracellular matrix (ECM) (e.g., a hydrogel) can be localized and provide a mechanically stable 3D structure for cell cultures. The microfluidic chip may also include a media channel where media can perfuse and sustain cell growth and function.

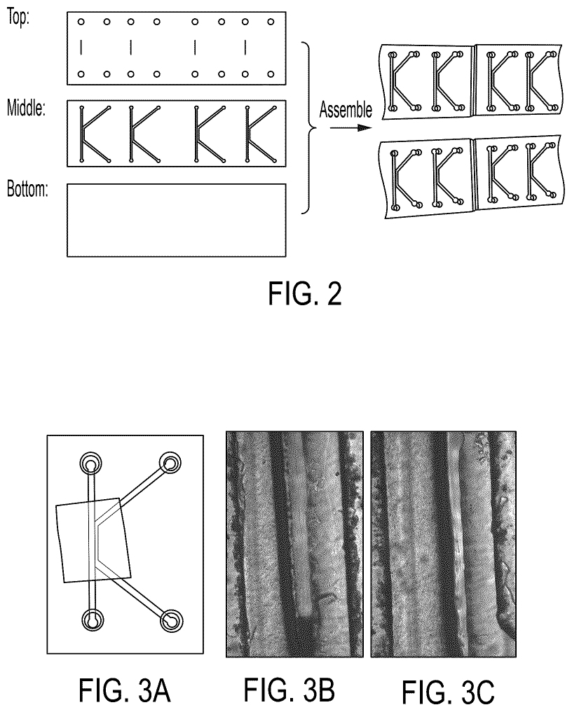

FIG. 2 shows one example design of a microfluidic chip or device, in accordance with one embodiment. In this example, pressure sensitive adhesives (PSA) were cut to K shapes and attached to the top plate of the device to act as microfluidic channel side walls. The top and bottom surfaces of the channels were made of thermoplastic polymers. A trench was patterned on the top plate of the chip and aligned with the PSA tapes to provide a physical barrier between the ECM channel and the medium channel in the common interconnect region. After the K shapes were attached to the top plate, protective liner and tapes in the ECM and medium channels were removed to expose three walls of the channels and the trench, which were then treated with corona plasma and coated with polymer solutions. The bottom plate was made of a different polymer material and was treated with corona plasma and coated with polymer solutions. The microfluidic chip was assembled after all the protective liners from the PSA was removed from the top piece and the top piece is bonded with the bottom plate.

In the example as shown in FIG. 2, the top plate used was PMMA, and the bottom plate was polystyrene. However, other thermoplastic materials such as COP, COC, and polycarbonate could be functionalized similarly. These materials showed similar behavior and could also be used as substrates.

Surface functionalization of the microfluidic channels was performed as follows. The substrate with the PSA tapes was first subjected to corona plasma treatment at 30 W with a chamber pressure of 1 mbar for 330 seconds, then the exposed microfluidic channels (both ECM and media channel) and the trench were coated with 1% (w/v) PVP (MW: ˜10 k) by either immersing the entire corona treated substrates into ˜15 mL of PVP solution (“dip coating”), or casting ˜20 microliters PVP solution on top of the corona treated surface (“drop casting”). These were then incubated at room temperature for 15 minutes. After drying at room temperature by compressed air, the coated substrates were placed on a hotplate at 65° C. for 30 minutes to remove residue water moisture, completing surface coating.

Next, the remaining protective liner of the PSA was removed, and the substrate was ready to be bonded with another substrate, with or without the K shaped PSA tapes, to form the microfluidic chips. In some cases, multiple microfluidic chips can be multiplexed on the same substrate to make the microfluidic slide or plate.

After functionalization, the surfaces at the common interconnect region between the ECM channel and media channel was hydrophilic throughout. The trench at the top plate cut through the middle of the common interconnect region and provided a physical barrier between the ECM and the medium channels so when hydrogel was injected into the ECM channel from one end, it flowed along the ECM channel without crossing the trench or entering into the media channel.

The hydrophilicity of the ECM channel may facilitate the flow of fluid containing the hydrogel precursor into the ECM channel after deposition at one end of the channel. In addition, the surface hydrophilicity of the media channel may facilitate media perfusion in some cases, which may allow either pumpless or pump-driven perfusion of media. The trench in the common interconnect region of the ECM and media channels may provide physical separation of the channels to facilitate gel localization when injected into the channels. In some cases, the size of the trench can be optimized such that after hydrogel is solidified in the ECM channel, there is no physical separation between the hydrogel and the perfused medium flow in the media channel, e.g., to allow free exchange of material in the molecular level.

In some cases, hydrophilic polymers or other coating materials may be used to coat the microfluidic channels. The polymer coating may be selected to be relatively homogenous and provide sufficient hydrophilicity to facilitate the flow and support for the hydrogel. In some cases, the polymer may affect cell distribution in the hydrogel and/or on the hydrogel/media interface. For example, polyvinylpyrrolidone (PVP) and polyethylene glycol (PEG) may be used as a coating. These are charge-neutral polymers which may be conducive to evenly distribute cells in the hydrogel. In some cases, ECM attachment factor solution (FAS) and/or polylysine may be used as coating materials. For example, they may be used as charged materials which may attract more cells to the coating, e.g., for certain specific applications where cell adhesion to surface is desired.

To coat the microfluidic channels after treatment, techniques such as dip coating or drop casting can be used. The coating method, volume, and concentration of the coating solution can be varied, e.g., depending on the desired thickness of the polymer coating based on application needs.

The length of the channel of the microfluidic chips may depend on the size of the substrate, e.g., a slide or plate, etc. In some cases, there may be inlets and outlets for the hydrogel and for media perfusion. For instance, as non-limiting examples, for microfluidic chips that are multiplexed onto a 384 well plate equivalent microtiter plate, the channel length may be selected to be 9 mm, the channel width can vary from 200 micrometers to 1000 micrometers, and/or the channel height can vary from 120 micrometers to 480 micrometers. The aspect ratio of the channel width vs height may be used to control hydrogel flow and localization within the ECM channel. In some cases, the aspect ratio may be used to control cell conditions in the hydrogel or on the hydrogel/media interface.

The dimension of the trench may be useful to control the performance of the microfluidic chips. In some cases, the trench may be selected to be wide enough to provide sufficient separation between the channels. In some cases, the trench may be selected to allow physical separation between the solidified hydrogel and the perfusion medium flow.

Different kind of hydrogel can flow into and stay localized within the ECM channel. For instance, collagen I can be used, e.g., from different sources such bovine and rat. Other hydrogels such as other collagens, Matrigel®, Gel-MA, etc. can also be used.

### Example 2

This example shows a non-limiting design of a microfluidic chip in FIG. 2. In this example, the chip has three layers. The top layer is a 2.6 mm thick PMMA (polymethylmethacrylate) slide with holes and trenches created by a CO₂ laser. The trench dimension is approximately 6 mm (length)×0.2 mm (width)×2.6 mm (height). The middle layer is a 0.24 mm thick double-sided pressure sensitive adhesive (PSA) with channel shapes cut by a UV-laser. The bottom layer is a polystyrene slide. Both the ECM channel and the media channel of the PSA layer were removed before attaching it with the top layer. During this step, the holes and trenches were vertically aligned such that the trench was positioned between the ECM channel and medium channel. The assembled top/middle layer and bottom layer were coated by polymer separately before being assembled into the final device, shown as a photograph with eight individual repeat units.

FIG. 3 shows the localization and stability of a hydrogel within an ECM channel. All of the channel surfaces in this example, including the trench surfaces, were coated with 1% PVP after corona treatment, and the resulting hydrogel solution (0.4% collagen I) only flowed through the ECM channel. After gelation, the solidified collagen I remained intact without notable volume change.

FIG. 3A is a photograph showing a microfluidic chip with a trench. The chip contains localized collagen I after gelation at 37° C. The total channel width (ECM+medium channel) was 800 micrometers. FIGS. 3B and 3C are optical images of the resulting channel after gelation. There is a distinct difference in the light transmittance in the ECM channel and media channels, indicating the presence and absence of collagen gel in the ECM and media channels, respectively. The boundary is consistent with the position of the trench, indicating the trench acted as the physical barrier to prevent collagen solution from flowing into the medium channel.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An article, comprising:
a substrate defining a first microfluidic channel having a first inlet and a first outlet, and a second microfluidic channel having a second inlet and a second outlet, the first microfluidic channel and the second microfluidic channel positioned parallel and separated by a trench within a common interconnect region positioned between their respective inlets and outlets.

2. The article of claim 1, wherein the trench is substantially straight.

3. The article of claim 1, wherein the trench has a cross-sectional dimension of at least 10 micrometers.

4. (canceled)

5. The article of claim 1, wherein the trench has a maximum length of at least 3 mm.

6. The article of claim 1, wherein the trench has a depth of at least 10 micrometers.

7. (canceled)

8. The article of claim 1, wherein the common interconnect region has a length defined where the first microfluidic channel and the second microfluidic channel are positioned parallel of at least 3 mm.

9. The article of claim 1, wherein the first microfluidic channel and the second microfluidic channel are in fluidic contact within the common interconnect region.

10-14. (canceled)

15. The article of claim 1, wherein the second microfluidic channel comprises a first bend between the second inlet and the common interconnect region, and a second bend between the common interconnect region and the second outlet.

16-23. (canceled)

24. The article of claim 1, wherein the first microfluidic channel comprises a hydrogel.

25. The article of claim 24, wherein the hydrogel fills the first microfluidic channel but not the second microfluidic channel.

26. The article of claim 24, wherein the hydrogel partially fills the common interconnect region.

27. The article of claim 24, wherein at least 20% of any cross-section of the common interconnect region is not filled with the hydrogel.

28. The article of claim 24, wherein at least a portion of the hydrogel in the first microfluidic channel is exposed to the second microfluidic channel.

29-35. (canceled)

36. The article of claim 1, wherein at least a portion of the first microfluidic channel contains a coating material positioned on the first microfluidic channel.

37-38. (canceled)

39. The article of claim 36, wherein the coating material comprises poly(ethylene glycol).

40. (canceled)

41. The article of claim 36, wherein the coating material is hydrophilic.

42-48. (canceled)

49. The article of claim 1, wherein at least a portion of the substrate defining the common interconnect region is substantially transparent.

50. The article of claim 1, wherein the substrate defines a plurality of repeat units, wherein at least some of the repeat units are defined by the first microfluidic channel, the second microfluidic channel, and the common interconnect region.

51. (canceled)

52. The article of claim 50, wherein the substrate comprises at least 5 repeat units.

53. The article of claim 50, wherein the substrate has dimensions of (75 mm+/−2 mm)×(26 mm+/−2 mm).

54. The article of any one of claim 50, wherein the substrate has dimensions of (128 mm+/−5 mm)×(85 mm+/−5 mm).

55. An article, comprising:
a substrate defining a first microfluidic channel and a second microfluidic channel, the first microfluidic channel containing a hydrogel and the second microfluidic channel being free of hydrogel, the first microfluidic channel and the second microfluidic channel positioned parallel within a common interconnect region such that an interface is present within the common interconnect region between the hydrogel in the first microfluidic channel and the second microfluidic channel, the substrate further defining a trench positioned adjacent the interface.

56-73. (canceled)

74. A method, comprising:
providing a substrate defining a first microfluidic channel having a first inlet and a first outlet, and a second microfluidic channel having a second inlet and a second outlet, the first microfluidic channel and the second microfluidic channel positioned parallel within a common interconnect region positioned between their respective inlets and outlets; and
passing a fluid through the first microfluidic channel from the inlet towards the outlet, through the common interconnect region, wherein the fluid is prevented from entering the second microfluidic channel via a trench in a wall of the common interconnect region.

75-94. (canceled)
